(19) United States
(12) Patent Application Publication
(10) Pub. No.: US 2006/0229617 A1
(43) Pub. Date: Oct. 12, 2006

(54) Intramedullary devices and methods of deploying the same

(75) Inventors: Nimrod Meller, Kiryat Tivon (IL); Avraham Shekalim, Nesher (IL); Nir Cohen, Tel Aviv (IL)

Correspondence Address: DR. MARK FRIEDMAN LTD.;C/O BILL POLKINGHORN, 9003 FLORIN WAY, UPPER MARLBORO, MD, 20772, US

(73) Assignee: OrthoMechanics Ltd.

(21) Appl. No.: 11/232,919

(22) Filed: Sep. 23, 2005

Related U.S. Application Data
(60) Provisional application No. 60/655,884, filed on Feb. 25, 2005.

(51) Int. Cl. A61F 2/30

(52) U.S. Cl. 606/62

(57) ABSTRACT

Internally locking intramedullary nails or devices and methods of deploying internally locking intramedullary nails are disclosed. According to some embodiments, the nail includes a cannulated sleeve, a plurality of anchoring elements, and one or more extension mechanisms for outwardly extending anchoring elements to anchor against movement along an elongated axis of the sleeve. Exemplary methods include but are not limit to methods of securing an internally locking intramedullary nail within a fracture bone, methods of effecting a reamed deployment of an internally locking intramedullary nail, and methods of modifying the extend to which anchoring elements are extended to effect dynamization useful for inducing bone growth. According to some embodiments, the internally locking intramedullary device includes a first extension mechanism operative to outwardly extend at least one anchoring element of a first set of anchoring elements through respective radially openings in the sleeve at an oblique position facing one end of the sleeve to anchor against movement in a longitudal direction and a second extension mechanism independent of or decoupled from the first extension mechanism operative to outwardly extend at least one anchoring element of a second set of anchoring elements through respective radially openings in the sleeve at an oblique position facing the opposite end of the sleeve to anchor against movement in the opposite longitudal direction. According to some embodiments, the intramedullary device includes a differential extension mechanism operative to extend anchoring elements through respective radial openings such that an increase in displacement of individual said anchor elements of first and second groups of anchoring elements generated by operation of the differential extension mechanism is distributed between the first and second groups as a function of resistance encountered by the first and second groups of anchoring elements. Some embodiments of the present invention provide hip prosthetic implant for replacing the proximal portion of a femur, where the implant includes at least one deformable clamping element for outwardly engaging surrounding bone tissue to anchor a stem portion of the implant within the intramedullary canal.

### CROSS-REFERENCE TO RELATED APPLICATIONS

This application claims priority to U.S. Provisional Application Ser. No. 60/655,884, filed on Feb. 25, 2005, which is incorporated herein by reference in its entirety.

### FIELD OF THE INVENTION

The invention relates to surgical devices for fixing broken bones and to hip implant devices.

### BACKGROUND OF THE INVENTION

Intramedullary Devices

Bone fractures are treated by realigning the broken bone fragments and immobilizing them in their formerly healthy positions relative to one another until the body causes the bone to heal and restore its structural integrity. Immobilization or fixation of the segments is accomplished by the use of rigid devices that span the fracture site and are located either external to the body or internally on the bone surface or inside the medullary canal.

Intramedullary fixation devices, which are indicated primarily in the fracture of long tubular bones, offer substantial advantages over external devices or those that are attached to the external surface of the bone. Such advantages include restoring functional rehabilitation of the limb within a relatively short time, freedom from the need for multiple surgical incisions to insert and remove holding pins and screws, reduced fluoroscopy, reduced incidence of infection and, unlike external holding devices, they are not easily susceptible to inadvertent movement.

Unfortunately, despite their advantages, many intramedullary fixation devices known in the art are not completely satisfactory. A discussion of shortcomings of prior art intramedullary devices is provided in U.S. Pat. No. 6,575,973 of one of the present inventors, the disclosure of which is incorporated herein by reference in its entirety. Generally speaking, it is revealed that many currently known devices fail to securely engage the inside of the medullary canal, thus providing only limited lateral support. Unfortunately, this can allow for potential rotational and migratory movement of the bone fragments relative to one another.

U.S. Pat. No. 6,575,973 discloses an internal fixation device including an elongate tubular sleeve and at least two anchoring elements oriented such that the outward displacement of one anchoring element anchors the engaged bone fragment against movement in one longitudinal direction, and the outward displacement of the other anchoring element anchors the engaged bone fragment against movement in the opposite longitudinal direction.

By locking to proximal and distal fragments of the broken bone, the device of U.S. Pat. No. 6,575,973 connects the fragments of broken bone, allowing patients to bear weight on the bone at an early stage, which facilitates the healing of the bone without shortening and without rotation of bone fragments.

Unfortunately, U.S. Pat. No. 6,575,973 does not disclose a method of effecting a reamed deployment of the device, and the suitability of the device in the context of reamed deployment is unclear.

From both a mechanical and a biological point of view, medullary reaming is particularly beneficial in improving the performance of implants. Specifically, reaming expands the medullary canal so that larger diameter implants can be inserted. These larger diameter implants are less likely to fail. In fact, certain fractures require over-reaming so that larger implants can be used. Without reaming, the surgeon must use a “best guess” estimate when selecting the diameter of the implant. The medical literature contains numerous case studies reporting the adverse consequences of an inaccurate estimate. Reaming provides a direct measurement of the diameter of the medullary canal, and thereby allows for the selection of an implant that precisely fills the canal. As a result, the stability of the fracture site is enhanced by achieving endosteal contact. When implants do not fill the medullary canal, load sharing between the implant and the bone is decreased. This increases the load that is transferred to the implant and promotes both implant failure and stress shielding of the bone.

There is an ongoing need for methods of effecting reamed deployments of intramedullary fixing devices that securely anchor to a bone fragment and are capable of holding both fragments of a broken bone in place without exerting compressive force upon them.

Furthermore, the device disclosed in U.S. Pat. No. 6,575,973 includes a single mechanism for extending anchoring elements which move in tandem. The extension mechanism includes a threaded shaft, and the distance at which anchoring elements are extended by the shaft depends on the threading pitch. If desired, the threads of the two ends of the shaft may have different pitches such that the rotation of the shaft produces different displacements of the nuts, and thereby of their respective anchoring elements at the opposite ends of the shaft. The unequal thread pitch allows the anchoring elements of U.S. Pat. No. 6,575,973 to protrude at different rates.

There are many situations where this property is desirable, such as when implanting a device into a conical or other wise irregular sections of bone. Thus, the physician can select a device with thread pitch characteristics appropriate for the specific geometry of the bone section, and then implant the selected device. Once implanted, the relative rates at which specific anchoring elements protrude are predetermined by the threat pitch properties of the shaft. Unfortunately, this approach is not always feasible, since it is not always clear to the physician before implant what the appropriate ratio should be. Furthermore, in many situations the desired device with the specified thread pitch properties might not be readily available.

Thus, it would be desirable to have an intramedullary fixing device that securely anchors to a bone fragment where the geometry of device anchoring can be controlled by the attending physician during or after surgery. Furthermore, it would be desirable to have an intramedullary fixing device that securely anchors to a bone fragment where the geometry of device anchoring can be determined by bone geometry as well as the local mechanical properties of the bone in which the device is anchored. Such as device would be particularly useful in conical or other wise irregular sections of bone.

Prosthetic Hip Implant

There is an ongoing medical need for devices and methods for securing with the intramedullary canal prosthetic hip implants for replacing the proximal portion of femurs. In particular, it is desirable that such devices would be adjustable by a physician after implant to induce bone growth near the stem portion of the hip implant.

The following US Patents disclose potentially relevant background art. The disclosure of the listed US Patents is incorporated herein by reference: U.S. Pat. No. 5,849,004 U.S. Pat. No. 5,976,139, U.S. Pat. No. 6,183,474, U.S. Pat. No. 6,443,954, U.S. Pat. No. 6,488,684, U.S. Pat. No. 6,648,889, and U.S. Pat. No. 6,695,844

### SUMMARY OF THE INVENTION′

The aforementioned needs are satisfied by several aspects of the present invention.

It is now disclosed for the first time a method of effecting a reamed deployment of an internally locking intramedullary nail within a fractured bone. The presently-disclosed method includes the steps of (i) inserting a guide wire into a canal of the bone, (ii) inserting an elongated sleeve having a plurality of radial openings into the canal of the bone such that the sleeve passes along the guide wire, (iii) removing the guide wire from the elongated sleeve, (iv) outwardly extending a first set of at least one anchor element through respective radial openings in an oblique position facing one end of the sleeve to anchor against movement in a longitudal direction, and (v) outwardly extending a second set of at least one anchor element through respective radial openings in an oblique position facing the opposite end of the sleeve to anchor against movement in the opposite longitudal direction.

Although it is not a requirement that the outward extending of the first and second set are carried out sequentially, in some embodiments the outward extending of the first and second set are carried out sequentially.

According to some embodiments, extension of at least one set of anchoring elements includes substantially simultaneously extending a plurality of anchoring elements.

According to some embodiments, at least one step of extending includes (i) deploying a shaft coupled to an anchor element within the elongated sleeve, and (ii) engaging the shaft to outwardly extend the anchor element.

According to some embodiments, the engaging includes rotating the shaft within the sleeve.

According to some embodiments, the shaft is threaded, at least one anchoring element is coupled to the shaft via a nut engaged to the threading, and the rotation of the elongated shaft longitudally displaces an inner end of the coupled anchoring element.

According to some embodiments, the longitudal displacement causes the coupled anchoring element to engage an inclined surface to outwardly displace an outer end of the coupled anchoring element through its respective radial opening.

It is now disclosed for the first time a method of securing an internal fixation device within a fractured bone. The presently disclosed method includes (i) inserting an elongated sleeve having a plurality of radial openings into the canal of the bone, (ii) outwardly extending a first set of at least one anchor element through respective radial openings in an oblique position facing one end of the sleeve to anchor against movement in a longitudal direction, and (iii) following the extending of the first set of anchor elements, outwardly extending a second set of anchor elements through respective radially openings in an oblique position facing the opposite end of the sleeve to anchor against movement in the opposite longitudal direction.

According to some embodiments, the first extending includes the steps of (i) providing a first shaft coupled to the first set of anchoring elements within the sleeve, and (ii) engaging the first elongate shaft to outwardly extend the first set of anchoring elements within the sleeve. According to some embodiments, the second extending includes providing a second shaft coupled to the second set of anchoring elements within the sleeve and engaging the second shaft to outwardly extend the second set of anchoring elements within the sleeve.

According to some embodiments, at least one of the first and the second engaging includes rotating a respective shaft.

According to some embodiments, the first and second shafts are decoupled from each other.

According to some embodiments, the first and second elongated shaft are independently rotatable within the sleeve.

It is now disclosed for the first time method of fixing a fractured bone. The presently disclosed method includes the steps of (i) inserting into a canal of the bone an elongated sleeve having a radial opening on a proximal side and a radial opening on a distal side of the sleeve, (ii) through each radial opening outwardly extending anchor elements to anchor against longitudal movement such that a proximal anchor element is disposed in an oblique position facing one end of the sleeve and a distal anchor element is disposed in an oblique position facing the opposite end of the sleeve, (iii) waiting time to allow the bone to at least partially heal, and (iv) at least partially retracting only said proximal anchor element to allow axial play between fragments of the bone.

According to some embodiments, the outward extending of the distal anchoring element includes engaging a first shaft coupled to the distal anchoring element, and the outward extending of said proximal anchoring element includes engaging a second shaft coupled to the proximal anchoring element.

According to some embodiments, the first and second shafts are decoupled from each other.

According to some embodiments, the first and second shafts are independently rotatable within the sleeve.

According to some embodiments, retracting includes further engaging the second shaft.

It is now disclosed for the first time an internally locking intramedullary device particularly useful for securing bone fragments,. The presently disclosed device includes (i) an elongate tubular sleeve including plurality of radial openings for insertion into the medullary canal of the bone fragments to be secured and (ii) a plurality of anchoring elements, a first set of at least one anchoring element coupled to a first extension mechanism operative to outwardly extend at least one anchoring element of the first set of anchor elements through respective radial openings at an oblique position facing one end of the sleeve to anchor against movement in a longitudal direction, a second set of at least one anchor element coupled to a second extension mechanism decoupled from the first extension mechanism operative to outwardly extend at least one anchoring element the second set through respective radial openings at an oblique position facing the opposite end of the sleeve to anchor against movement in the opposite longitudal direction.

According to some embodiments, at least one extension mechanism includes a shaft rotatably movable within the sleeve and coupled to a respective set of anchoring elements such that the anchoring element extends outwardly upon rotation of the shaft within the sleeve.

According to some embodiments, the shaft is maintained at a longitudally fixed position within said sleeve during said rotation.

According to some embodiments, said shaft includes at least one included surface outwardly deflecting and extending a said anchoring element.

According to some embodiments, at least one respective radial opening of said first set is disposed on a proximal end, defined to be either the one end or the opposite end, of the elongated sleeve, and at least one respective radial opening of the second set is disposed on the distal end of the elongated sleeve.

According to some embodiments, each of the first and second set include at least two anchoring elements, and the first anchoring mechanism is operative to outwardly extend at least one element of the first set through respective radial openings at an oblique position facing the opposite end of the sleeve to further anchor against movement in the opposite longitudal direction, and the second anchoring mechanism is operative to outwardly extend at least one element of the second set through respective radial openings at an oblique position facing the one end of the sleeve to further anchor against movement in the longitudal direction.

It is now disclosed for the first time an internally locking intramedullary device particularly useful for securing bone fragments. The presently disclosed device includes (i) an elongated sleeve including a plurality of radial openings for insertion into the medullary canal of the bone fragments to be secured, and (ii) a plurality of anchoring elements coupled to a differential extension mechanism operative to outwardly extend each anchor element through a said radial opening such that an increase in displacement of individual the anchor elements of first and second groups of the anchoring elements generated by operation of the differential extension mechanism is distributed between the first and second groups as a function of resistance encountered by the first and second groups of anchoring elements.

According to some embodiments, the differential extension mechanism includes a rotatable and longitudally movable shaft within the sleeve coupled to the anchor elements of the first and second groups, the anchoring element of the first group being extendable by rotation of the shaft, the anchoring elements of the second group being extendable by longitudal motion of the shaft, wherein resistance encountered by at least one anchor element of the first group imposes longitudal movement upon the shaft thereby outwardly extending the second anchor element.

According to some embodiments, at least one anchoring element of the second group is constrained from rotation within the sleeve.

According to some embodiments, each radial opening includes an inclined surface for deflecting an anchor element outwardly as the anchor element moves longitudally with respect to the sleeve.

According to some embodiments, at least one anchor element of the first group is outwardly extended through a first radial opening at an oblique position facing one end of the sleeve to anchor against movement in a first longitudal direction, at least one anchor element of the second group is outwardly extended through a second radial opening at an oblique position facing the opposite end of the sleeve to anchor against movement in the opposite longitudal direction It is now disclosed for the first time an implant for replacing the proximal portion of a femur. The presently disclosed implant includes (i) a head member having a spherical portion configured for positioning into a hip socket, (ii) an elongated stem portion adapted for insertion into the intramedullary canal of the femur joined to the head member, and (iii) at least one deformable clamping element for outwardly engaging surrounding bone tissue upon relative linear displacement of two ends of the deformable elongated clamping element towards each other to produce an outward displacement of at least a medial portion of deformable clamping element thereby securing the elongated stem portion within the intramedullary canal

According to some embodiments, a deformable clamping element is elongated and substantially parallel to the axis of the elongated stem portion.

According to some embodiments, a proximal end of the clamping element is substantially located at a proximal end of the elongated stem portion and a distal said end of said clamping element is substantially located at a distal end of said elongated stem portion.

According to some embodiments, an axial surface of the stem portion includes at least one axially elongated slot and at least a portion of the clamping element is adapted to fit through the elongated slot.

According to some embodiments, a local deformation property of a clamping element varies to at least partially locally to determine an outward displacement of the clamping element.

According to some embodiments, the local deformation property is selected from the group consisting of a local thickness of the clamping element, a local cross section of the clamping element, and a local elasticity of the clamping element.

According to some embodiments, a clamping element includes proximal, distal and the medial portions, and at least a portion of the medial portion is less deformable than both the proximal and distal portions.

According to some embodiments, the implant further includes a linear displacement mechanism configured to linearly displace a first end of the clamping element thereby contributing to the relative linear displacement of the two ends of the clamping element.

According to some embodiments, the elongated stem section includes an axial bore having a threaded portion, and a plurality of the clamp elements are substantially parallel to each other and joined together at the first end to form a clamp element array, and the linear displacement mechanism includes an externally threaded section of the clamping array engaged with the threaded portion.

According to some embodiments, a second end of the clamping element is attached to the elongated stem portion thereby substantially fixing an axial position of the end of the clamping element.

According to some embodiments, the linear displacement mechanism includes a lock for substantially fixing an axial position of the first end of the clamping element.

According to some embodiments, the linear displacement mechanism includes a linear movable element connected to the first end of the clamp element via a compressible element and a relationship between a linear displacement of the linear movable element and a linear displacement of the first end of the clamp is determined at least in part by compressive properties of the compressive element.

According to some embodiments, the compressive element includes a spring.

These and further embodiments will be apparent from the detailed description and examples that follow.

### BRIEF DESCRIPTION OF THE DRAWINGS

FIG. 1 provides an exploded view of an exemplary intramedullary nail according to some embodiments of the present invention.

FIG. 2 provides a cross section view of the intramedullary nail.

FIGS. 3 and 4 provide drawing of the intramedullary nail during different stages of a reaming procedure.

FIGS. 5-11 provide illustrations of a self locking intramedullary nail according to exemplary embodiments of the present invention.

FIG. 12 provides an illustration of an intramedullary nail deployed in an irregularly shaped bone.

FIG. 13 provides an illustration of a system useful for treating hip fractures

FIG. 14 provides an illustration of a hip prosthesis or implant.

FIG. 15 provides an illustration of the internal clamping device FIG. 16 provides an isometric view of the prosthesis body of the prosthetic device.

FIG. 17 provides a view of a hip prosthesis according to exemplary embodiments of the present invention.

### DETAILED DESCRIPTION OF THE INVENTION

The present invention will now be described in terms of specific, example embodiments. It is to be understood that the invention is not limited to the example embodiments disclosed. It should also be understood that not every feature of the implantable devices and methods of treatment described is necessary to implement the invention as claimed in any particular one of the appended claims. Various elements and features of devices are described to fully enable the invention. It should also be understood that throughout this disclosure, where a process or method is shown or described, the steps of the method may be performed in any order or simultaneously, unless it is clear from the context that one step depends on another being performed first.

It will now be described an intramedullary nail to connect the fragments of a broken bone. The nail is locked to the proximal and distal fragments of the broken bone, allowing patients to bear weight on the bone early, thereby facilitating the healing of the bone without concomitant shortening or rotations of the fragments.

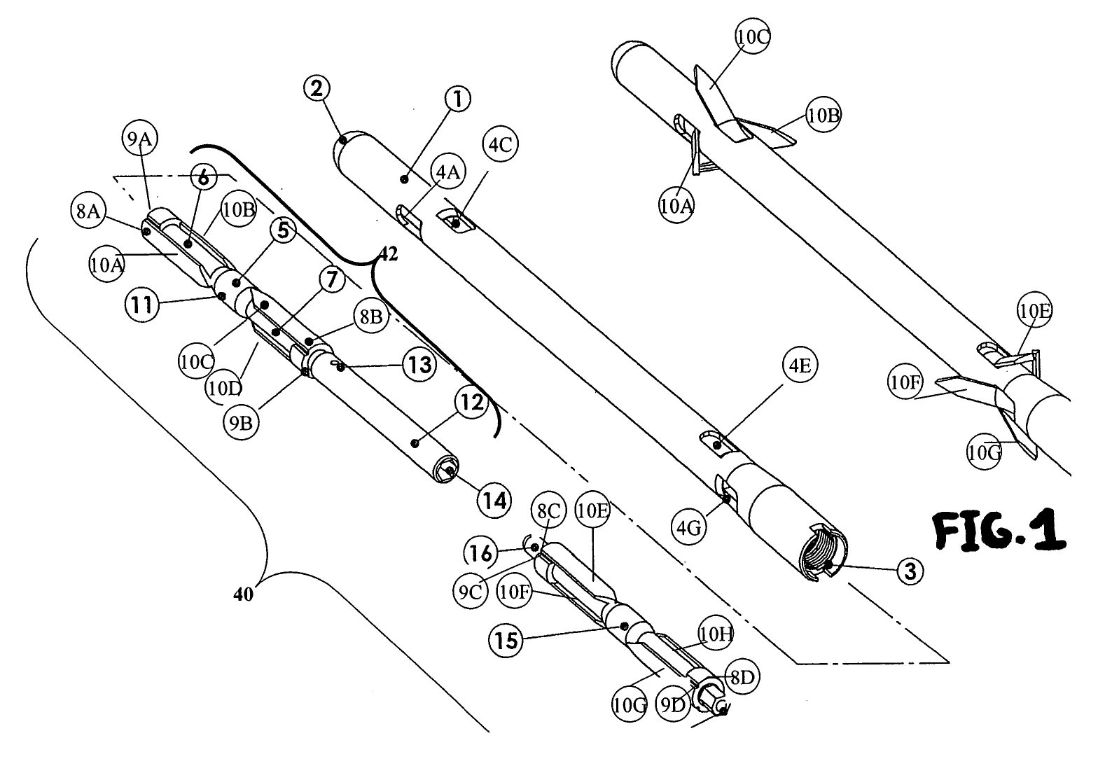

FIG. 1 provides an exploded view of an exemplary intramedullary nail according to some embodiments of the present invention. Thus, as illustrated in FIG. 1 the intramedullary nail includes a sleeve or cannulated pin 1 having plurality of radial openings 4 through which anchoring elements 10 protrude. Optionally, the sleeve 1 is curved to fit the radius of a bone such as the femur.

Situated along the axis of the elongated sleeve 1 is a shaft assembly 40, including a distal shaft 5, a midshaft 12 and a proximal shaft 15. It is noted that the distal shaft 5, midshaft 12 and proximal shaft 15 are free to rotate within the elongated sleeve 1. The anchoring elements 10 are coupled to elements of the shaft assembly such that rotation of the shaft assembly extends 10 the anchoring elements through the radial openings 4 of the sleeve 1.

The anchoring elements 10 are cantilever beams, possibly with a sharp end, and are provided as parts of anchoring members 8. As shown in FIGS. 1-3, each anchoring member 8 includes two anchoring elements 10 and a threaded nut portion 9 with threads corresponding to the threaded portion of the shaft assembly 40 on which the nut portion 9 is fitted.

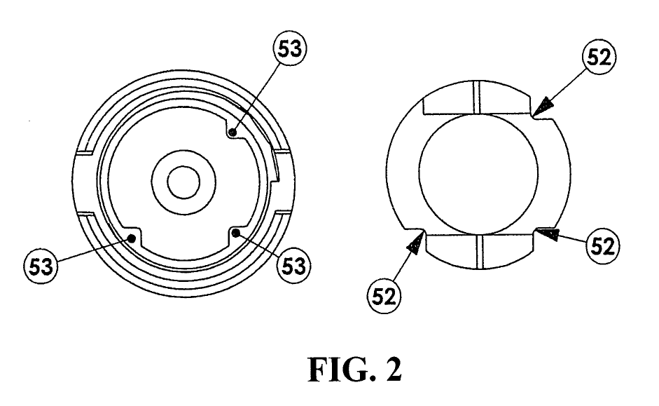

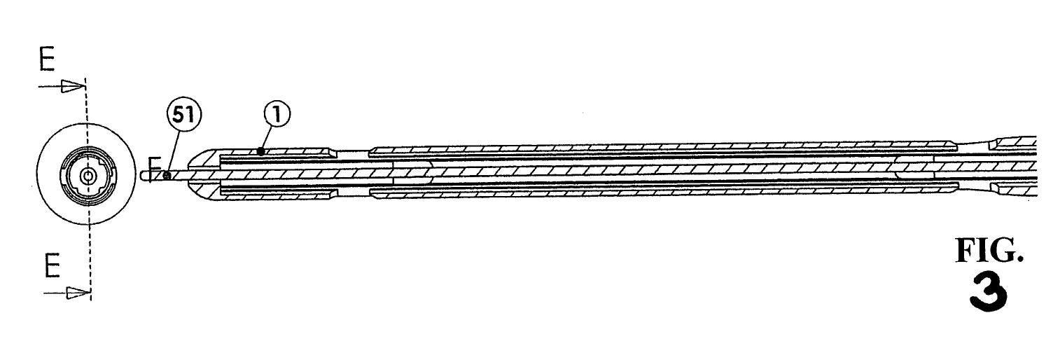

In contrast to the shaft assembly 40 which can rotate relative to the sleeve 1, the anchoring members 8 do not enjoy rotational freedom relative to the sleeve 1. FIG. 2 provides a cross section view of the intramedullary nail. The locking mechanism includes grooves the anchoring elements 10 which fit into slots of the rail 53 of the sleeve 1 thereby preventing rotation.

Referring once again to FIG. 1, the elongated tubular sleeve 1 includes a distal end 2, which is rounded and is adapted to be inserted within the medullary canal of a bone, and a proximal end 3 which is open. The proximal end (3) is capable of being rigidly attached to insertion and extraction mechanisms via threaded portion and key. There are rails on the inside of the sleeve 1 as depicted in the cross section (see FIG. 2A).

As illustrated in FIG. 1, there are a total of eight radial openings 4, four disposed distally (4A-4D) and four disposed proximally (4E-4H, though it will be appreciated that the device as disclosed in the figures could be modified to allow few than or more than the displayed number of radial openings. It is noted that at the distal end certain anchor elements are disposed through a respective radial opening 4 in an oblique position facing the distal end of the sleeve (e.g. 10C, 10D) and certain anchor element are disposed through a respective radial opening 4 in an oblique position facing the proximal end of the sleeve (e.g. 10A, 10B). Similarly, at the proximal end certain anchor elements are disposed through a respective radial opening 4 in an oblique position facing the distal end of the sleeve (e.g. 10F, 10H) and certain anchor element are disposed through a respective radial opening 4 in an oblique position facing the proximal end of the sleeve (e.g. 10E, 10G).

The shaft assembly includes a distal shaft 5, a midshaft 12 and a proximal shaft 15. The distal shaft 5 includes a threaded distal portion 6 and a threaded proximal portion 7, where the distal portion is threaded in a clockwise direction and the proximal portion is threaded in a counterclockwise direction (or vise versa). Between the threaded portions there is a distal shaft center section 11 which is of larger diameter than the threads, and merges the threaded portions via two conical sections, one conical section on the proximal side and one conical section on the distal side. The threaded pitch may be different between the two threaded portions or similar. A different pitch allows the anchors to protrude at different rate.

The proximal end 13 of the distal shaft 5 is attached to the midshaft 12 which is a simple cylinder. Any method of fixating the distal shaft 5 to the midshaft 12 known in the art is appropriate, including but not limited to pin, welding, gluing and the like. In some embodiments, the distal shaft 5 is fixated to the midshaft 12 after anchoring members 8A and/or 8B have been threaded onto the distal shaft 5. The proximal end of the midshaft 12 is fitted with a hexagonal bore 14.

The proximal shaft 15 is similar to the distal shaft, but has a rounded distal end 16. Thus, the proximal shaft 15 includes a threaded distal portion 47 and a threaded proximal portion 48, where the distal portion is threaded in a clockwise direction and the proximal portion is threaded in a counterclockwise direction (or vise versa). Between the threaded portions there is a distal shaft center section 15 which is of larger diameter than the threads, and merges the threaded portions via two conical sections, one conical section on the proximal side and one conical section on the distal side. The threaded pitch may be different between the two threaded portions or similar. A different pitch allows the anchors to protrude at different rate.

When inserted into the midshaft bore 14, the proximal shaft 15 is free to rotate while remaining concentric to the midshaft bore 14. The proximal end 17 of the proximal shaft 15 is shaped to attach rigidly to an insertion instrument called the distal introducer.

It is noted that the intramedullary nail of FIGS. 1-4 can be inserted into intramedullary canal using either a reamed procedure or an undreamed procedure.

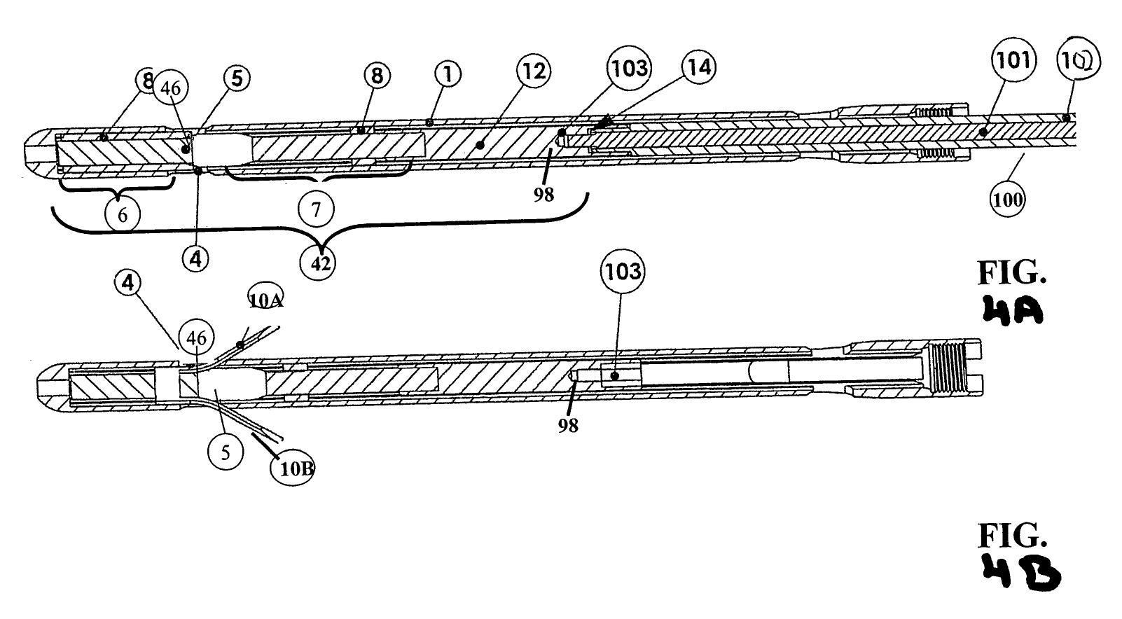

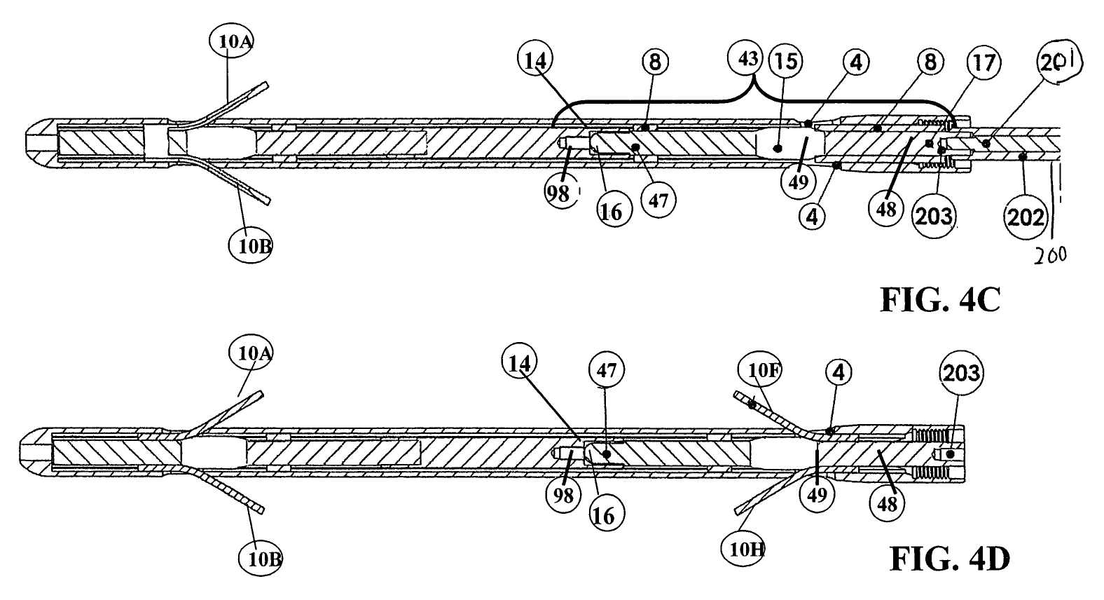

In a typical reamed procedure (FIGS. 3, 4A-4D), a hole is drilled in one end of the long bone, and then a guide wire 51 is inserted into the bone to allow for reducing of the fracture over the guide wire. After insertion of the wire 51, the intramedullary canal of the bone is reamed by a series of reamers to a desired inside diameter.

After the reaming, the sleeve 1 is inserted over the guide wire 51 into the bone (not shown). According to some embodiments, at the time of insertion of the sleeve 1, it is cannulated with the entirety of the shaft assembly 40 outside of the sleeve 1. Subsequently, upon removal of the guide wire 51, the distal shaft assembly 42 is inserted into the sleeve 1 (FIG. 3). The distal shaft assembly 42 includes the distal shaft 5, the midshaft 12, and one anchor member 8A including a threaded nut 9A and two anchor members 10A-B, where the distal shaft 5 is attached to the midshaft 12.

As illustrated in FIG. 4A, the distal shaft assembly 42 is inserted with a distal introducer 100. The distal introducer 100 includes an inner rod 101 and an outer distal introducer sleeve 102. It is noted that the inner rod 101 is threaded at the distal end. The distal introducer sleeve 102 attaches to the threaded introducer bore 98 of the midshaft 12, thereby locking the distal introducer 100, and more specifically distal end 103 of the inner rod 101, to the midshaft bore 14. Once the distal introducer 100 is rigidly locked to the midshaft 12, the distal introducer 100 can be used to apply a torque to the shaft assembly 40 and inter alia to the distal shaft 5. Optionally, the introducer can be locked axially, to allow an attending physician to remove the distal shaft.

Because the distal anchoring member 8A and the distal anchoring elements 10A, 10B are prevented from rotating, application of the torque to the distal shaft 5 causes the threaded nut 9A of the distal anchoring member 8A to advance in the proximal direction, thereby moving the distal anchoring elements 10A, 10B of the distal anchoring member 8A in the proximal direction. This motion causes the distal anchoring elements 10A, 10B of the distal anchoring member 8A to engage the distal ramp portion 46 of the distal conical section 5, deforming the distal anchoring elements 10A, 10B and inducing outward motion of the distal anchoring elements 10A, 10B through the distal radial openings 4A, 4B as shown in FIG. 4B.

The degree of outward motion through the distal radial openings 4A, 4B is determined at least in part by the geometric and material properties of the anchoring elements, openings and ramps. The force in the direction of the movement causes the anchoring elements to penetrate the bone and thus lock the distal part of the nail into the bone.

In some embodiments, after locking the distal anchoring elements 10A, 10B into the bone the distal introducer 100 is disengaged and removed. The proximal locking phase is similar to the distal locking phase, though the actual extension mechanism operative to outwardly extend proximal anchoring elements 10F, 10H is independent from the extension mechanism operative to outwardly extend distal anchoring elements 10A, 10B.

FIG. 4B illustrates the introduction into the sleeve 1 of the proximal shaft assembly including the proximal shaft 15 and proximal anchor member 8D. The proximal anchor member 8D includes the nut portion 9D and two anchoring proximal elements 10F, 10H.

As illustrated in FIG. 4C, the proximal shaft assembly 43 is inserted with a proximal introducer 200. The proximal introducer 200 includes an inner rod 201 and an outer proximal introducer sleeve 202. It is noted that the rod inner 201 is threaded at the distal end. The rounded end 16 of the proximal shaft 15 is disposed in the hexagonal bore 14 of the midshaft 12. Because of the shape disparity between the rounded end 16 and the hexagonal midshaft bore 14, the distal shaft assembly 42 and the proximal shaft assembly 43 do not rotate in tandem.

The tip of the proximal introducer 200 has a key 203 shaped to fit into the bore of the proximal end 17 of the proximal shaft 15, and once engaged, the proximal introducer can be rotated to apply a torque to the proximal shaft 15.

Because the proximal anchoring member 8D and the proximal anchoring elements 10F, 10H are prevented from rotating, application of the torque to the proximal shaft 15 causes the threaded nut 9D of the proximal anchoring member 8D to advance in the proximal direction, thereby moving the proximal anchoring elements 10F, 10H of the proximal anchoring member 8D in the distal direction. This motion causes the proximal anchoring elements 10F, 10H of the proximal anchoring member 8D to engage the proximal ramp portion 49 of the proximal conical section 15, deforming the proximal anchoring elements 10F, 10H and inducing outward motion of the distal anchoring elements 10F, 10H through the proximal radial openings 4F, 4H as shown in FIG. 4D.

The degree of outward motion through the proximal radial openings 4F, 4H is determined at least in part by the geometric and material properties of the anchoring elements, openings and ramps. The force in the direction of the movement causes the anchoring elements to penetrate the bone and thus lock the proximal part of the nail into the bone.

Some medical studies have indicated that a controlled amount of axial play between the fragments of the bone may be beneficial to the healing process. The free play may be introduced at some time after installation of the intramedullary nail, by whole or partially removing the locking between the device and the bone fragment at one end of the nail.

This can be achieved by a minor surgical procedure carried out some time, e.g. hours, days, weeks or months after installation of the intradmedullary nail. In some embodiments, after installation of the device of FIGS. 1-4, the proximal introducer 200 is re-engaged with the proximal shaft 15, and a torque is applied to the proximal shaft 15 in a direction such that the proximal anchoring elements 10F, 10H at least partially retract. That is correct.

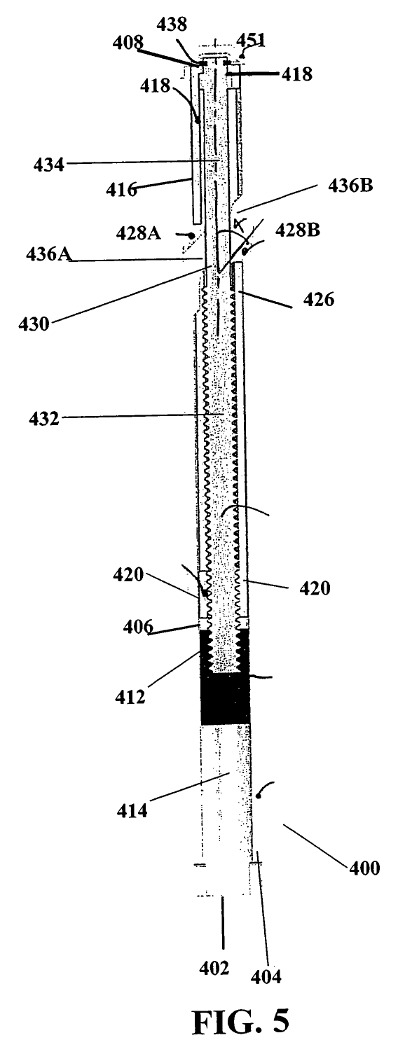

FIGS. 5-11 provide illustrations of a self locking intramedullary nail 400 according to exemplary embodiments of the present invention. The self locking intramedullary nail 400 includes four main components or assemblies: a cannulated pin or sleeve 404, an internal shaft 430, a proximal anchor member 420, and a distal anchor member 418.

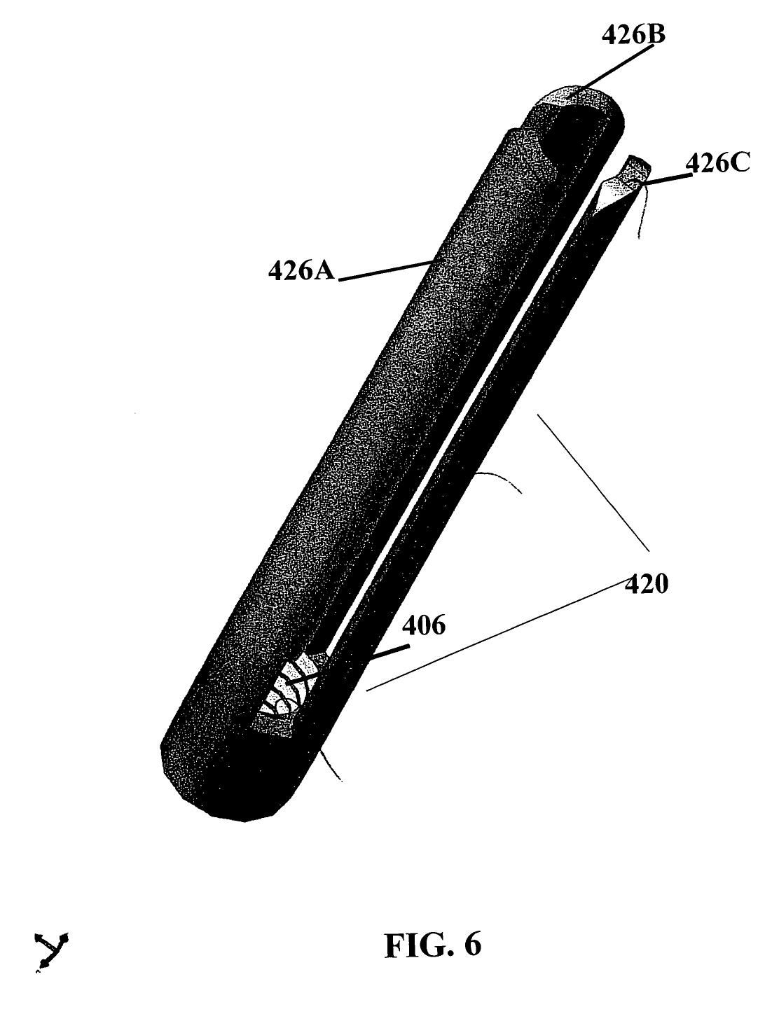

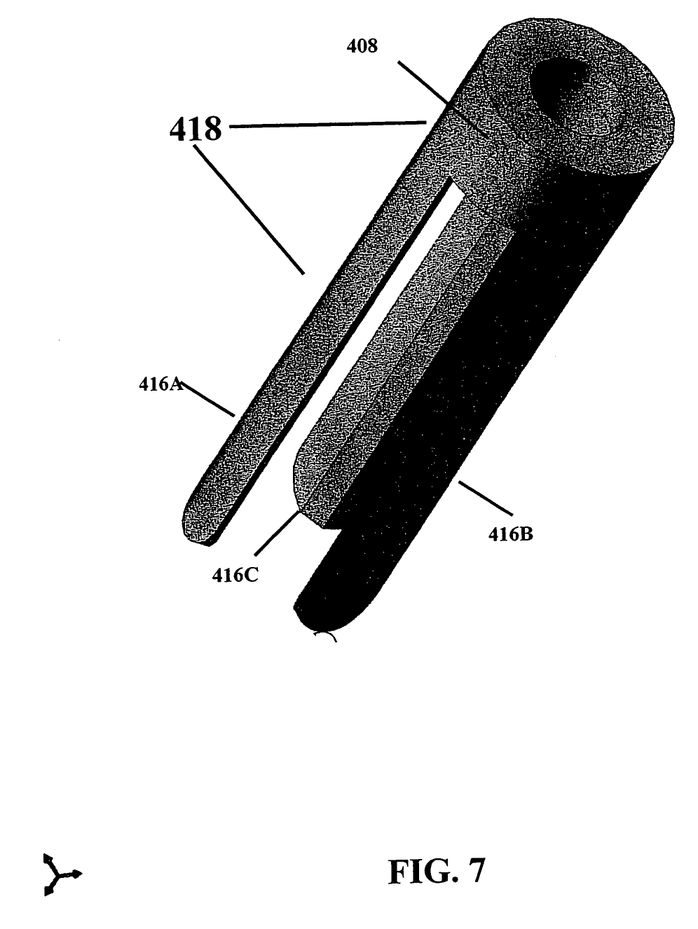

FIG. 6 provides an illustration of an exemplary proximal anchor member 420 or blade bundle according to some embodiments of the present invention. As shown in FIG. 6, the proximal anchor member 420 includes an internally threaded nut portion 406 and three leaves or blades or anchoring elements 426, though it is appreciated that fewer than three or more than three anchoring elements 426 are appropriate. FIG. 7 provides an illustration of an exemplary distal anchor element 418 according to some embodiments of the present invention. As shown in FIG. 7, the distal anchor member 418 includes a shoulder portion 408 and three blades or anchoring elements 416, though it is appreciated that fewer than three or more than three blades 416 are appropriate.

There are no specific limitations on the physical characteristics of the proximal 426 and/or distal 416 anchoring elements. In some embodiments, one or more proximal 426 and/or distal 416 anchoring elements are constructed from an elastic material (e.g. spring steel). Furthermore, it is noted that as illustrated in FIGS. 5-7 the proximal anchoring elements 426 are longer than the distal anchoring elements 416. In some embodiments, the end of at least one proximal 426 and/or distal 416 anchoring element is sharp and the anchoring element is a spike. Alternatively, the end of the anchoring element is blunt, wherein the specific properties of the anchor element are selected according to the application. The section and/or thickness of any anchoring element can be varied to control elastic and plastic deformation properties.

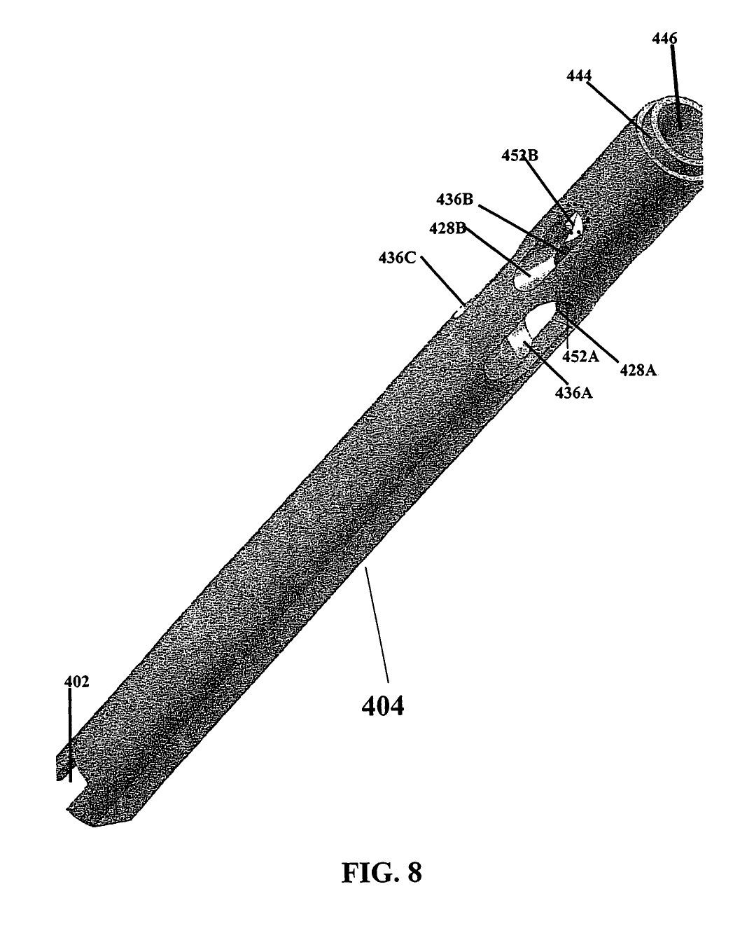

FIG. 8 provides an isometric view of the pin or sleeve 404 into which the internal shaft 430 as well as anchor members 418 and 420 is inserted. As shown in FIG. 8, the sleeve includes openings at both the proximal 402 and distal 446 ends. In some embodiments, the distal anchor member 418 is inserted into the pin or sleeve 404 through the distal opening 446, and after inserting the distal anchor member 418, the distal end of the sleeve is closed off with cap 438 which is welded to or snapped onto or otherwise attached to the distal end of the sleeve 404. As illustrated in FIG. 8, the sleeve includes a sleeve retainer ring 444 adapted to receive the cap 438. Optionally, the cap 438 is rounded (not shown) to ease insertion into the intramedullary canal.

The sleeve 404 includes a set of six circumferentially and equidistantly arrayed radial openings or slots 436 through which anchoring elements may protrude upon operation of the device, as described below. It is noted that each radial opening includes a ramp or inclined surface 428 and a longitudal engaging of the ramp 428 by an anchoring element converts longitudal motion into radial outward motion of the anchoring element, as will be described below. Each ramp 428 has a slope or angle relative to the longitudal axis of a sleeve 404, and the value of the angle may be specified in accordance with the specific application. Furthermore, it is noted that each radial opening 436 includes wedge 452 within the radial opening 436 which serves to prevent the distal anchoring member 418 (or blade assembly) from rotating relative to the sleeve 404. It is noted that there are three such wedges, radially disposed, separating three windows.

It is noted that the ramps 436 of a first set of three radial openings 436 face in the opposite longitudal direction of the ramp 436 of a second set of three other radial openings. When the anchoring elements (416 and 426) are deployed through the radial openings 436, the proximal anchoring elements 426 thus protrude through the first set of radial openings while the distal anchoring elements 416 protrude through the second set of radial openings.

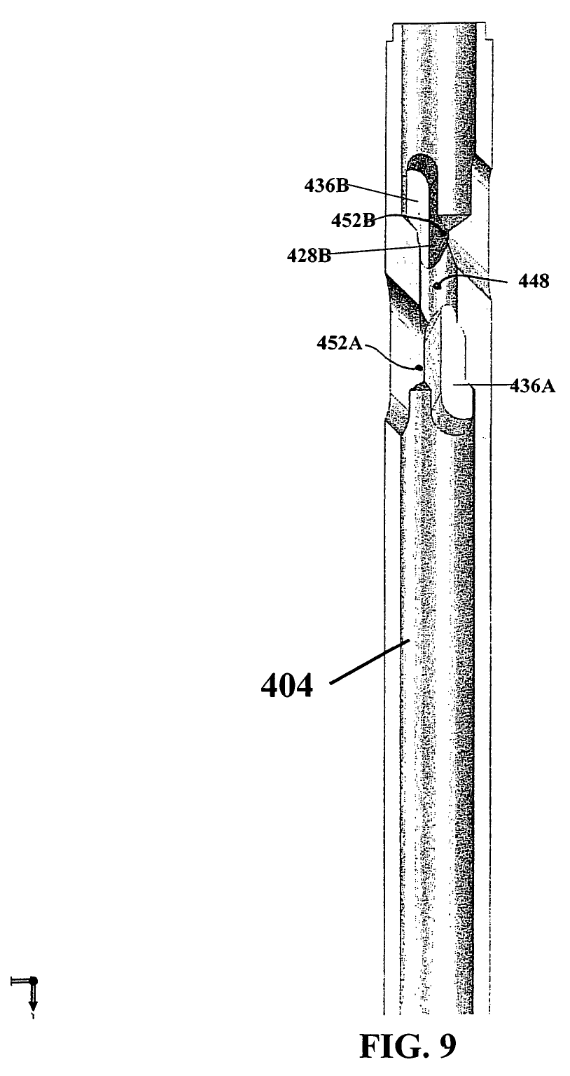

FIG. 9 provides another illustration of the sleeve 404.

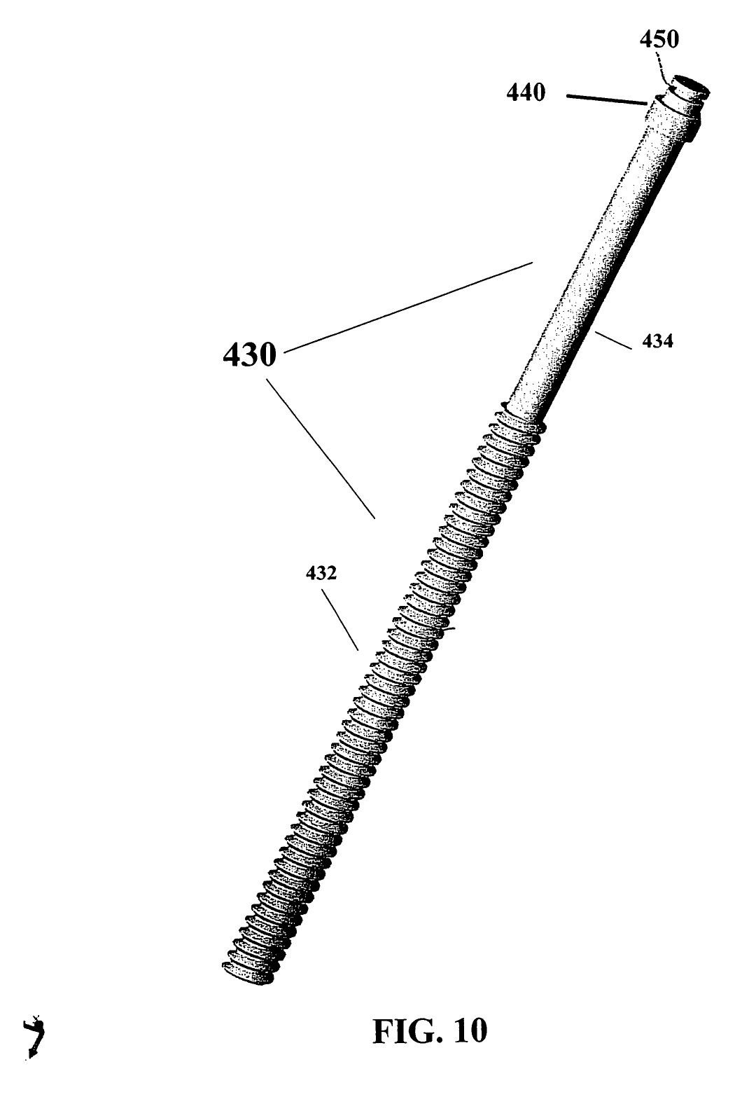

FIG. 10 provides an illustration of the inner shaft 430 according to some embodiments of the present invention. The inner shaft 430 includes an externally threaded section 432 at the proximal end and an unthreaded section 434 at the distal end. The internally threaded nut portion 406 of the proximal anchoring member 420 is coupled to the externally threaded section 432 of the inner shaft 430 such that rotation of the inner shaft 430 induces longitudal motion of the proximal anchoring member 420 This is made possible by the wedges 452 of FIG. 9 which substantially prevent rotational motion of the distal anchoring member 418.

The distal end of the inner shaft 430 includes a grove 450 for accepting the retainer ring 451 (not shown in FIG. 10). There is no specific limitation on the retainer ring, and in some embodiments it is a simple off the shelf “c-ring” or “snap-ring” which is used to mate components onto the shaft 430 so that they rotate about the shaft 430 but cannot translate axially relative to the shaft. The ring 451 is mated to the groove 450 on the shaft 430.

Thus, according to some embodiments, the distal anchoring member 418 is installed through the distal opening 446 of the sleeve 404 onto the inner shaft 430 such that the retainer ring 451 of the distal anchoring member 418 mates with the groove 450 of the inner shaft 430. It is noted that when the retainer ring 408 rests in the groove 450 this effectively prevents longitudal motion of the distal anchoring member 418 relative to the inner shaft 430.

Although longitudal the distal anchoring member 418 is axially locked to the inner shaft 430 according to exemplary embodiments of FIGS. 5-11, it is noted that, according to some embodiments, the distal anchor element is free to rotate relative to the inner shaft 436 but not relative to the sleeve 404.

Referring again to FIG. 5, attached to the inner shaft 430 is a coupling 412 for receiving an external driving device such as a screwdriver (not shown), where the coupling 412 is attached to the inner shaft 430 by, for example, by welding or by an adhesive glue material or by a mechanical fastener. To engage the coupling 412, the driving device is inserted into the axial bore 402 of the sleeve 404 though a proximal opening 402. It is noted that according to some embodiments, the axial shaft 430 rotatable and to translatable within the sleeve 404. Thus, engaging the proximal surface of the coupling 412 with the driving device allows for rotation and/or translation of the inner shaft 430 within the sleeve 404. It noted that the coupling 412 is not a limitation of the present invention. Alternatively, the inner shaft 430 lacks the coupling 412 and instead has a keyed hole (for example, a square or hexagonal bore).

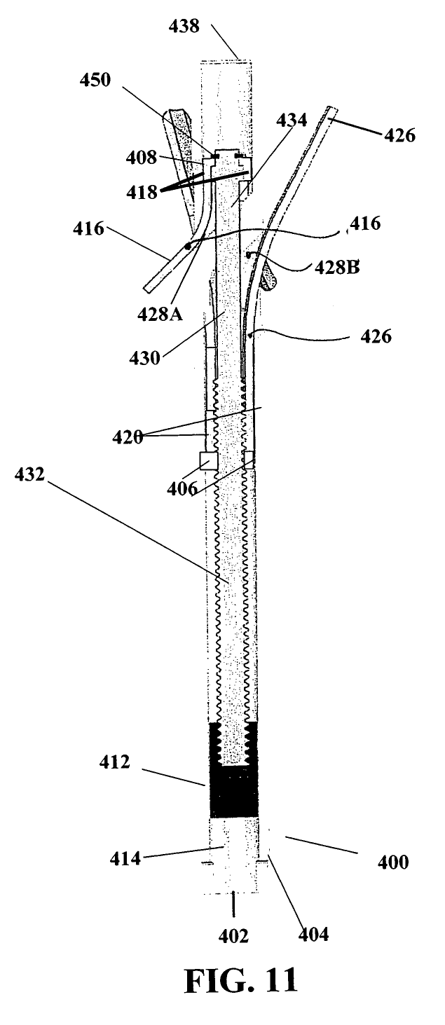

FIG. 11 provides an illustration of the device of FIG. 5 wherein both proximal 426 and distal 418 anchoring elements are deployed through respective radial openings 436. It is noted that proximal 428 and distal 418 anchoring elements are not necessarily deployed in tandem, and that in some embodiments, the extension mechanism operative to extend one or more proximal 428 anchoring elements through the respective radial openings 438 is separate from or independent of or decoupled from the extension mechanism operative to extend one or more proximal 428 anchoring elements.

Thus, in the specific example of FIGS. 5-11, the distal anchor element 416 is deployed or outwardly extended by pulling the internal shaft 430 in the proximal direction, causing the distal anchor element 416 to engage the ramp 428A. Engagement of the ramp 428A causes axial motion of the distal anchor element 416 to be converted into outward motion away from central axis of the sleeve 404, thereby causing the distal anchor element 416 to protrude through the radial opening. Continued axial motion in the proximal direction of the inner shaft 430 causes the distal anchor element 416 to further extend outwardly.

The proximal anchoring elements 426 are deployed or outwardly extended by rotating the inner shaft 430. As externally threaded portion 432 of the inner shaft 430 rotated, the threaded nut portion 406 is engaged, causing the proximal anchor member 420, and more specifically the proximal anchoring elements 426 to longitudally advance towards the distal end of the sleeve 404. As the proximal anchoring elements 426 translate towards the distal end of the sleeve 404, they engage the ramp 428B which causes axial motion of the proximal anchor element 426 to be converted into outward motion away from central axis of the sleeve 404, thereby causing the proximal anchor element 426 to protrude through the radial opening. Continued axial motion in the distal direction of the inner shaft 430, driven by the rotational motion of the inner shaft 430, causes the proximal anchor element 416 to further extend outwardly.

According to a first mode of operation, it is noted that by rotating the inner shaft 430 only, while maintaining the inner shaft 430 at a longitudally fixed position relative to the sleeve 404, it is possible to extend and/or retract one or more proximal anchoring elements 426 without concomitantly extending and/or retracting one or more distal anchoring elements 416. In particular, according to this mode of operation, the nut 406 of the anchor member 404 advances in a proximal and/or distal direction relative to both the inner shaft 430 as well as the sleeve 404, which remain in a fixed longitudal relation to each other. This causes the anchoring elements 426 to advance in a distal and/or proximal direction, thereby inducing only outward extension and/or retraction of the anchoring elements 426 without influencing the deployment of the distal anchoring elements 416.

According to a second mode of operation, the inner shaft 430 is pulled in a proximal direction at a certain rate, while concomitantly the inner shaft 430 is rotated to longitudally advance the proximal anchoring elements 426 towards the distal end of the sleeve at the same rate that the inner shaft 430 advances in the proximal direction, causing the proximal anchor member 420, the nut 406 and the proximal anchoring elements 426 to maintain a fixed longitudal position relative to the sleeve 404. Thus, according to this second mode of operation, only one or more distal anchoring elements 416 are extended and/or retracted independent of any extension and/or retraction of any of the proximal anchoring elements 426.

Thus, it is noted that the device described in FIGS. 5-11 provides a first extension mechanism operative to outwardly extend one or more distal anchoring elements 416, and a second extension mechanism operative to outwardly extend one or more proximal anchoring elements 426 where the first and second extension mechanisms are independent of each other or decoupled from each other, and where only the distal anchoring elements and/or only the proximal anchoring elements are extended outwardly and/or retracted inwardly.

Nevertheless, it is stressed that the implementations of the first and second extension mechanisms as described in FIGS. 5-11 relate only to specific embodiments of the present invention, and are not intended as a limitation. The present invention is thus intended to encompass any device with any known extension mechanisms operative to extend and/or retract anchoring elements and not only the specific extension mechanism described in the examples. In particular, the present invention includes devices with a first extension mechanism is operative to extend a first anchoring element though a radial opening (e.g. a first radial opening) at an oblique position facing one end of the sleeve to anchor against movement in a longitudal direction, and second extension mechanism operative to extend a second anchoring element through a radial opening (e.g. a second radial opening) at an oblique position facing the opposite end of the sleeve to anchor against movement in the opposite longitudal direction. Thus, there is no specific limitation on the first and second extension mechanisms, and any known extension mechanisms for extending and/or retracting anchoring elements can be used in presently disclosed devices.

It is noticed that operating the device of FIGS. 5-11 using any combination of the first and second modes of operation allows for extension and/or retraction of the proximal 426 and/or distal 416 anchoring elements in tandem where a ratio between outward and/or inward motion of proximal and distal anchoring elements is in accordance with any predetermined ratio. The predetermined ratio can be achieved by appropriately rotating and translating the internal shaft 430 with external manipulation of the screwdriver engaged with the coupling 412.

A third mode of operating the device of FIGS. 5-11 will now be disclosed. According to this mode of operation, the inner shaft 430 is rotated with no concomitant external restriction imposed on the longitudal position of the inner shaft 430 relative to the sleeve. Thus, the turning of the inner shaft 430 forces the proximal anchoring 426 elements to translate in a distal direction, wherein upon encountering the ramp 428B the proximal anchoring elements 426 extend outwardly into the surrounding medium, e.g. bone tissue, wherein the proximal anchoring elements 426 encounter further resistance. The resistance encountered by the proximal anchoring elements 426 causes the internal shaft 430 to be reacted proximally relative to the sleeve 404, which, in turn, forces the distal anchoring elements 416 to longitudally translate in the proximal direction relative to the sleeve 404. Upon longitudal translation of the distal anchoring elements 416 towards the proximal end of the sleeve 404, the distal anchoring elements 416 encounter resistance from the ramp 436A as well as from the surrounding medium thus reacting the inner shaft 430 in the proximal direction.

Thus, according to this third mode of operation, imposing a torque on the inner shaft 430 results in outward movement of both the proximal 426 as well as the distal 416 anchor element through respective radial openings 436 in opposite longitudal directions. Furthermore, the set of anchoring elements of the proximal 426 and distal 416 anchoring elements that momentarily encounters the lesser resistance outwardly extends until a balance is reached. In this sense, according to the third mode of operation, the presently disclosed device of FIGS. 5-11 provides a differential extension mechanism operative to outwardly extend each anchor element (416 and 426) through a respective radial opening 436 such that an increase in displacement relative to the sleeve 404 of individual anchoring elements of first and second groups of anchoring elements, e.g. proximal anchoring elements 426 as those of the first group and distal anchoring elements 416 as those of the second group, generated by operation of the differential extension mechanism is distributed between the first and second groups as a function of total resistance encountered by the first and second groups of anchoring elements.

In some embodiments, the increase in displacement is a differential displacement or an infinitesimal increase in displacement.

In particular, the anchoring elements extend outwardly such that the total resistances encountered by the first group of anchoring elements (e.g. the proximal anchoring elements 426) is equalized with the total resistance encountered by the second group of anchoring elements (e.g. the distal anchoring elements 416). In order for these resistances to be equalized, it is noted that a given engagement of the differential extension mechanism extends respective anchoring elements of the first and second groups of anchoring elements variable distances in accordance with the resistances encountered by anchoring elements of the first and second groups. In some embodiments, anchoring elements of a group encountering a higher relative resistance extend at a slower rate than a given engagement of the differential extension mechanism extends respective anchoring elements of the first and second groups of anchoring elements variable distances in accordance with the resistances encountered by anchoring elements of the first and second groups. In some embodiments, the ratio between the increase in displacement between anchoring elements of the first and second group is linearly related to the ratio between resistance encountered by anchoring elements of the first and second groups.

Thus, unlike the first and second modes of operation, where the ratio between displacements of proximal and distal anchoring elements can be determined by the input forces and torques on the internal shaft, according to the third mode of operation, the ratio between the increase in displacement of the proximal and distal anchoring elements is determined by a ratio between resistances encountered by proximal and distal anchoring elements.

It is noted that the particular differential extension mechanism of FIGS. 5-11 is provided as an illustrating example, and any intramedullary nail including a differential extension mechanism operative to outwardly extend anchoring elements with these properties is within the scope of the present invention.

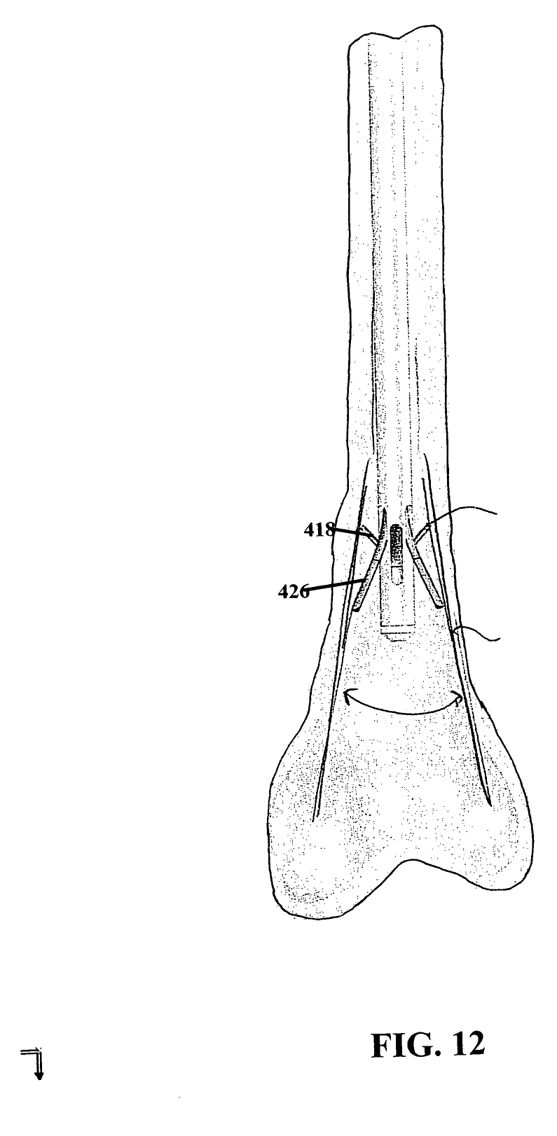

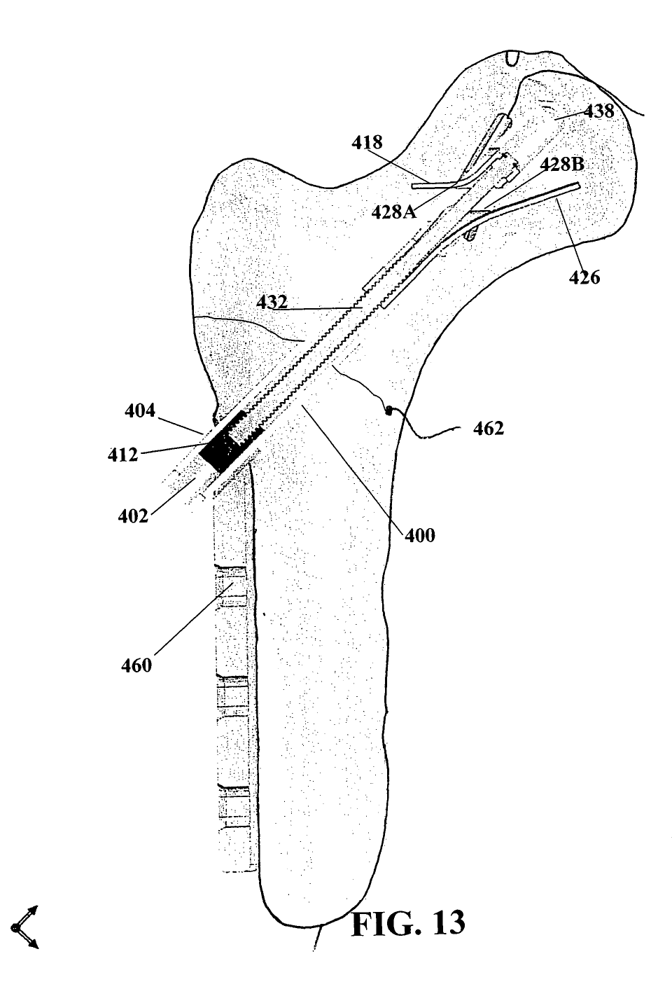

Not wishing to be bound by any particular theory, it is noted that devices wherein the increase in displacement of individual anchoring elements of first and second groups is determined by total resistance encountered by outwardly extending elements are useful in a number of applications. For example, in irregularly shaped bones, as illustrated in FIG. 12, it is desirable for particular anchoring elements to penetrate bone cortex in order to secure the intramedullary nail device. As illustrated in FIG. 12, the distance proximal and distal anchoring elements need to protrude through the radial opening in order to meet the cortex differs. As the device is installed and the anchoring elements are deployed, the respective clearance between the tips of anchoring elements and bone cortex is not always clear to the attending physician.

Thus, in one example related to the illustration of FIG. 12, after the intramedullary nail is inserted into the intramedullary canal, no anchoring elements protrude through any radial openings. Engagement of the differential extension mechanism outwardly extends both proximal and distal anchoring elements through the surrounding spongy bone. The distal anchoring elements reach the bone cortex first, and the resistance offered by the bone cortex is much greater than the resistance encountered by the proximal anchoring elements as they extend through spongy bone. Thus, when only the distal anchoring elements have reached the cortex, further engagement of the differential extension mechanism only outwardly extends the proximal anchoring elements. In this way, it is possible to further engage the extension mechanism until the tip of both the proximal and distal anchoring elements reach the bone cortex.

It is noted that the relative resistance encountered by proximal and/or distal anchoring elements, and hence the relative rate at which respective sets of anchoring elements outwardly extend depend upon the resistance encountered by the respective ramps and the surrounding bone tissue into which the anchoring elements extend. Thus, the relative resistances and the relative rate at which sets of anchoring elements extend depend upon a number of physical parameters, including but not limited to the incline angle of the ramp, the cross section and thickness of specific anchoring elements, and the material of which specific anchoring elements are constructed.

FIG. 13 provides an illustration of a system useful for treating hip fractures including a plate 460 and an intramedullary nail 400 as disclosed in FIGS. 5-11. The disclosed system is useful for treating fracture 462. In this specific example, distal anchoring elements 418 are less stiff than corresponding proximal anchoring elements 426, and as such, the resistance encountered by distal anchoring elements is reduced relative to the resistance encountered by proximal anchoring element. Thus, when engaging the differential extension mechanism, the rate at which distal anchoring element 418 outwardly extend relative to the rate of extension of proximal anchoring elements 426 is concomitantly increased, and the distal anchoring elements deform and deploy first with the benefit of compressing the fracture. When the distal anchoring elements 418 encounter the hard cortex, the proximal anchoring elements 426 will outwardly extend and deploy into the spongy bone. For the specific application of FIG. 13, the edges of the anchoring elements are blunt in order to avoid damaging the cortical shell of the bone.

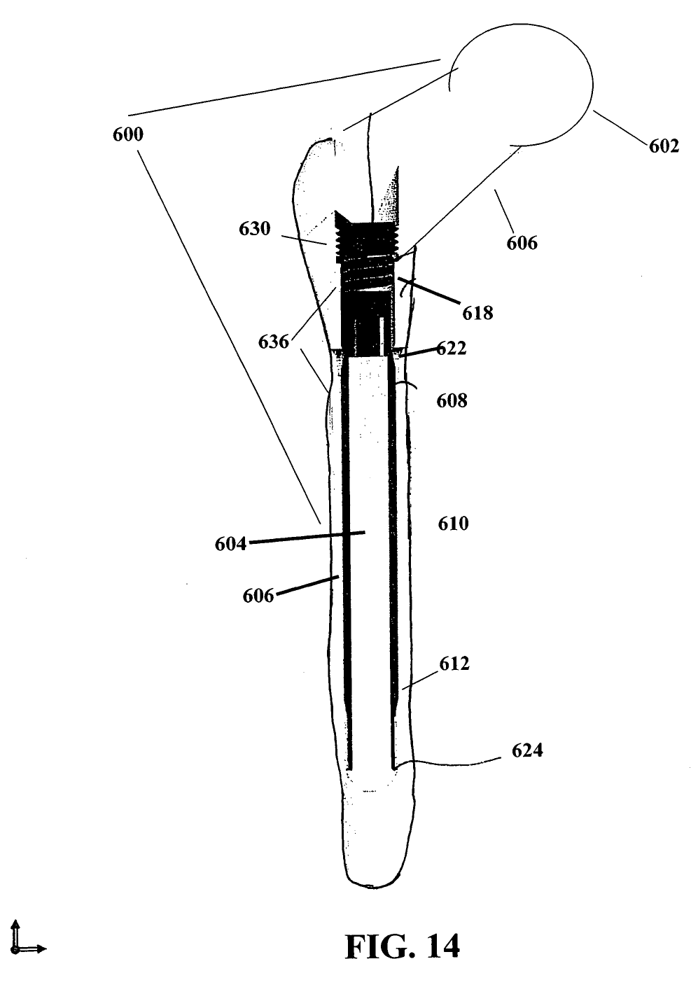

FIG. 14 provides an illustration of a hip prosthesis or implant 600 for implant into a headless femur according to exemplary embodiments of the present invention. The hip prosthesis includes a ball bearing 602 or head member having a spherical portion configured for positioning into a hip socket such as a natural or prosthetic hip socket, a neck portion 606, and an elongated stem portion 606 adapted for insertion into the intramedullary canal of the femur.

Furthermore, the device includes internal clamping device 636 including an array of at least one deformable internal clamping element 606. As shown in FIG. 14, each deformable clamping element is elongated and substantially parallel to the axis of the elongated stem portion 604. Furthermore, it is noted that the ends of the leaf or blades or internal clamping elements 606 are either blunt or sharp, depending on the specific application

Upon relative linear displacement of two ends (622 and 624) of linear clamping elements 606 towards each other, or in particular, when a proximal end 622 of the deformable internal clamping element 606 approaches distal end 624 of the internal clamping element 606, there is a bulging or outward displacement of at least a medial portion 610 of the internal clamping element 606. This bulge or outer displacement produces an outward force or outward pressure which outwardly engages surrounding bone (e.g. spongy bone and/or cortical bone), thereby securing the elongated stem 604 within the intramedullary canal. As shown in FIG. 14, the proximal end 622 of the deformable internal clamping element 606 is substantially located at the proximal end of the elongated stem portion 604 of the hip implant 600, while the distal end 624 of the deformable internal clamping element 606 is substantially located at the distal end of the elongated stem portion 604.

In some embodiments, a local deformation property of the internal clamping element 606 varies at least partially locally. Exemplary local deformation properties include but are not limited to a local thickness of the clamping element, a local cross section of the clamping element, and a local elasticity of the clamping element.

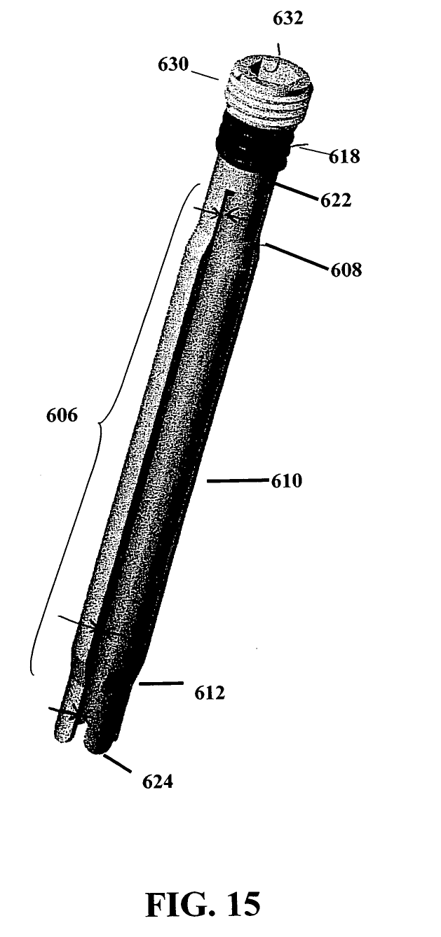

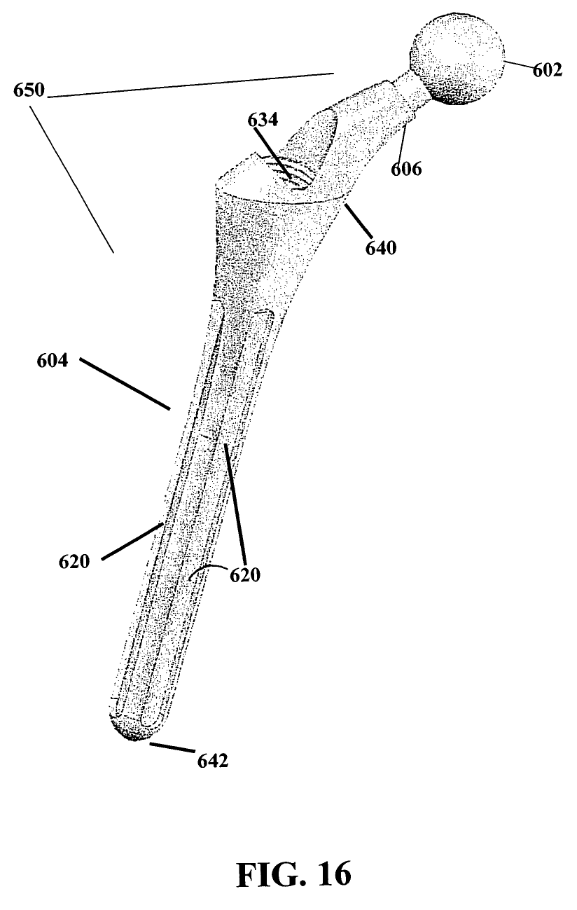

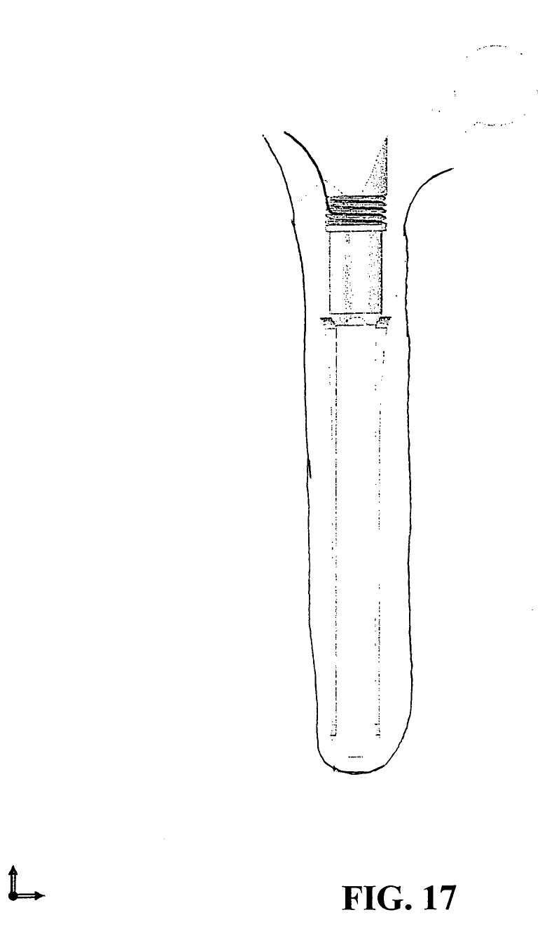

FIG. 15 provides an illustration of the internal clamping device 636 including an externally threaded preload adjustment screw 630 attached to spring 618 and the internal clamping element 606. It is noted that as illustrated in FIG. 15, the internal clamping element 606 includes proximal 608, medial 610, and distal 612 portions, where the medial 610 portion is thicker than both the proximal 608 and distal 612 portions. For the embodiment of FIG. 15, the medial 610 portion is thus less deformable than both the proximal 608 and distal 612 portions, and it is noted that most of the elastic deflection is localized near the ends (622 and 624) of the internal clamping element. The medial 610 portion is substantially not deformed, at least relative to the proximal 608 and distal 612 portions.

In some embodiments, the distal end 624 of the internal clamping element 606 is fastened to the elongated stem portion 604 of the hip prosthetic implant 600. Any fastening mechanism known in the art is appropriate for immobilizing the distal end 624 of the internal clamping element 606 on the elongated stem portion 604, including but not limited to mechanism fastening and welding.

With the distal end of the internal clamping element 606 immobilized, it is noted that by distally displacing the proximal end 622 of the internal clamping element 606 with the adjustment screw 614, the proximal end 622 is drawn closed to the distal end 624 thus outwardly deforming the internal clamping element 606 to secure the stem portion 604 of the prosthetic implant 600.

Towards this end, the internal clamping device 636 includes an externally threaded adjustment screw 630. When the adjustment screw 630 rotated, such as using a screwdriver inserted into the adjustment screw 630 axial bore, the externally threaded adjustment screw 630 interacts with the internally threaded implant stem axial bore 634 (see FIG. 16) located at the proximal end 640 of the stem portion, to displace the externally threaded adjustment screw 630 towards the distal end 642 of the stem portion 604.

The aforementioned linear displacement mechanism including the externally threaded adjustment screw 630 and the internally threaded axial bore 634 is provided as one specific example of a linear displacement mechanism or linear displacement device. Thus, it is noted that this should not be construed as a limitation, and any linear displacement mechanism or device is appropriate for the present invention.

As shown in FIG. 15, the adjustable screw 630, which is part of the linear displacement mechanism, is connected to the proximal end 622 of the clamping elements 606 via a compressible element, namely a spring 618. By adjusting the mechanical and/or compressive properties of the spring 618, it is possible to determine a relationship between linear displacement of the screw 632 and linear displacement of the proximal end 622 of the internal clamping elements 606. Furthermore, it is noted that the spring 618 provided in FIG. 15 is merely one example of a compressive element, and any compressive element is appropriate for the present invention.

It is noted that the degree of outward displacement or bulging of internal clamping elements 606 can be changed after installation of the device in the femur of the patient. In some applications, this allows for dynamization and for inducing bone growth to further anchor the device in the femur after implant.

FIG. 16 provides an isometric view of the prosthesis body 650 of the prosthetic device according to some embodiments of the invention. As illustrated in FIG. 16, the elongated stem section 604 includes a plurality of axially elongated slots 620 for storage of the clamp elements 606. Not wishing to be bound by any particular theory, it is noted that in some embodiments, the clamp elements 606 are placed within the slots 620 as the prosthetic device is implanted in the femur, and outwardly displaced using the linear displacement mechanism after implant.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

What is claimed is:

1) A method of effecting a reamed deployment of an internally locking intramedullary nail within a fractured bone, the method comprising:
a) inserting a guide wire into a canal of the bone;
b) inserting an elongated sleeve having a plurality of radial openings into the canal of the bone such that said sleeve passes along said guide wire;
c) removing said guide wire from said elongated sleeve;
d) outwardly extending a first set of at least one anchor element through respective radial openings in an oblique position facing one end of said sleeve to anchor against movement in a longitudal direction; and
e) outwardly extending a second set of at least one said anchor element through respective radial openings in an oblique position facing the opposite end of said sleeve to anchor against movement in the opposite longitudal direction.

2) The method of claim 1 wherein extension of at least said one set of anchoring elements includes substantially simultaneously extending a plurality of said anchoring elements.

3) The method of claim 1 wherein at least one said step of extending includes:
i) deploying a shaft coupled to a said anchor element within said elongated sleeve; and
ii) engaging said shaft to outwardly extend said anchor element.

4) The method of claim 3 wherein said engaging includes rotating said shaft within said sleeve.

5) The method of claim 4 wherein said shaft is threaded, at least one said anchoring element is coupled to said shaft via a nut engaged to said threading, and said rotation of said elongated shaft longitudally displaces an inner end of said coupled anchoring element.

6) The method of claim 5 wherein said longitudal displacement causes said coupled anchoring element to engage an inclined surface to outwardly displace an outer end of said coupled anchoring element through its respective radial opening.

7) A method of securing an internal fixation device within a fractured bone, the method comprising:
a) inserting an elongated sleeve having a plurality of radial openings into the canal of the bone;
b) outwardly extending a first set of at least one anchor element through respective said radial openings in an oblique position facing one end of said sleeve to anchor against movement in a longitudal direction; and
c) following said extending of said first set of anchor elements, outwardly extending a second set of said anchor elements through respective said radially openings in an oblique position facing the opposite end of said sleeve to anchor against movement in the opposite longitudal direction,

8) The method of claim 7 said first extending includes:
i) providing a first shaft coupled to said first set of anchoring elements within said sleeve; and
ii) engaging said first elongate shaft to outwardly extend said first set of anchoring elements within said sleeve, and said second extending includes:
i) providing a second shaft coupled to said second set of anchoring elements within said sleeve; and
ii) engaging said second shaft to outwardly extend said second set of anchoring elements within said sleeve,

9) The method of claim 8 wherein at least one engaging selected from the group consisting of said first and said second engaging includes rotating a respective said shaft.

10) The method of claim 9 wherein said first and second shafts are decoupled from each other.

11) The method of claim 8 wherein said first and second elongated shaft are independently rotatable within said sleeve.

12) A method of fixing a fractured bone, the method comprising:
a) inserting into a canal of the bone an elongated sleeve having a radial opening on a proximal side and a radial opening on a distal side of said sleeve;
b) through each said radial opening outwardly extending anchor elements to anchor against longitudal movement such that a proximal anchor element is disposed in an oblique position facing one end of said sleeve and a distal anchor element is disposed in an oblique position facing the opposite end of said sleeve;
c) waiting time to allow the bone to at least partially heal; and
d) at least partially retracting only said proximal anchor element to allow axial play between fragments of the bone.

13) The method of claim 12 wherein said outward extending of said distal anchoring element includes engaging a first shaft coupled to said distal anchoring element, and said outward extending of said proximal anchoring element includes engaging a second shaft coupled to said proximal anchoring element.

14) The method of claim 13 wherein said first and second shafts are decoupled from each other.

15) The method of claim 14 wherein said first and second shafts are independently rotatable within said sleeve.

16) The method of claim 13 wherein said retracting includes further engaging said second shaft.

17) An internally locking intramedullary device particularly useful for securing bone fragments, comprising:
a. an elongate tubular sleeve including plurality of radial openings for insertion into the medullary canal of the bone fragments to be secured; and
b. a plurality of anchoring elements, a first set of at least one said anchoring element coupled to a first extension mechanism operative to outwardly extend at least one said anchoring element of said first set of anchor elements through respective radial openings at an oblique position facing one end of said sleeve to anchor against movement in a longitudal direction, a second set of at least one said anchor element coupled to a second extension mechanism decoupled from said first extension mechanism operative to outwardly extend at least one said anchoring element of said second set through respective radial openings at an oblique position facing the opposite end of said sleeve to anchor against movement in the opposite longitudal direction.

18) The device of claim 17 wherein at least one said extension mechanism includes a shaft rotatably movable within said sleeve and coupled to a respective set of said anchoring elements such that said anchoring element extends outwardly upon rotation of said shaft within said sleeve.

19) The device of claim 17 wherein said shaft is maintained at a longitudally fixed position within said sleeve during said rotation.

20) The device of claim 19 wherein said shaft includes at least one included surface outwardly deflecting and extending a said anchoring element.

21) The device of claim 17 wherein at least one said respective radial opening of said first set is disposed substantially on a proximal end selected from said one end and said opposite end of said elongated sleeve, and at least one said respective radial opening of said second set is disposed substantially on said a distal end of said elongated sleeve.

22) The device of claim 17 wherein each of said first and second set include at least two said anchoring elements, and said first anchoring mechanism is operative to outwardly extend at least one said element of said first set through respective radial openings at an oblique position facing said opposite end of said sleeve to further anchor against movement in said opposite longitudal direction, and said second anchoring mechanism is operative to outwardly extend at least one said element of said second set through respective radial openings at an oblique position facing said one end of said sleeve to further anchor against movement in said longitudal direction.

23) The device of claim 17 wherein at least one said radial opening includes an inclined surface for outwardly deflecting and extending a said anchoring element.

24) An internally locking intramedullary device particularly useful for securing bone fragments, comprising:
a. an elongated sleeve including a plurality of radial openings for insertion into the medullary canal of the bone fragments to be secured; and
b. a plurality of anchoring elements coupled to a differential extension mechanism operative to outwardly extend each said anchor element through a said radial opening such that an increase in displacement of individual said anchor elements of first and second groups of said anchoring elements generated by operation of said differential extension mechanism is distributed between said first and second groups as a function of resistance encountered by said first and second groups of anchoring elements.

25) The device of claim 24 wherein said differential extension mechanism includes a rotatable and longitudally movable shaft within said sleeve coupled to said anchor elements of said first and second groups, said anchoring element of said first group extendable by rotation of said shaft, said anchoring elements of said second group extendable by longitudal motion of said shaft, wherein resistance encountered by at least one said anchor element of said first group imposes longitudal movement upon said shaft thereby outwardly extending said second anchor element.

26) The internal fixation device of claim 25 wherein said at least one anchoring element of said second group is constrained from rotation within said sleeve.

27) The internal fixation device of claim 24 wherein said each radial opening includes an inclined surface for deflecting a said anchor element outwardly as said anchor element moves longitudally with respect to said sleeve.

28) The internal fixation device of claim 24 wherein at least one said anchor element of said first group is outwardly extended through a first said radial opening at an oblique position facing one end of said sleeve to anchor against movement in a first longitudal direction, at least one said anchor element of said second group is outwardly extended through a second said radial opening at an oblique position facing the opposite end of said sleeve to anchor against movement in the opposite longitudal direction.

29) An implant for replacing the proximal portion of a femur, the implant comprising:
a) a head member having a spherical portion configured for positioning into a hip socket;
b) an elongated stem portion adapted for insertion into the intramedullary canal of the femur joined to said head member; and
c) at least one deformable clamping element for outwardly engaging surrounding bone tissue upon relative linear displacement of two ends of said deformable elongated clamping element towards each other to produce an outward displacement of at least a medial portion of said deformable clamping element thereby securing said elongated stem portion within said intramedullary canal

30) The implant of claim 29 wherein a said deformable clamping element is elongated and substantially parallel to the axis of said elongated stem portion.

31) The implant of claim 30 wherein a proximal said end of said clamping element is substantially located at a proximal end of said elongated stem portion and a distal said end of said clamping element is substantially located at a distal end of said elongated stem portion.

32) The hip prosthesis of claim 31 wherein said an axial surface of said stem portion includes at least one axially elongated slot and at least a portion of said clamping element is adapted to fit through said elongated slot.

33) The implant of claim 29 wherein a local deformation property of a said clamping element varies to at least partially locally determine a said outward displacement of said clamping element.

34) The implant of claim 33 wherein said local deformation property is selected from the group consisting of a local thickness of said clamping element, a local cross section of said clamping element, and a local elasticity of said clamping element.

35) The implant of claim 33 wherein a said clamping element includes proximal, distal and said medial portions, and at least a portion of said medial portion is less deformable than both said proximal and distal portions.

36) The implant of claim 30 further comprising:
d) a linear displacement mechanism configured to linearly displace a first said end of said clamping element thereby contributing to said relative linear displacement of said two ends of said clamping element.

37) The implant of claim 36 wherein said elongated stem section includes an axial bore having a threaded portion, and wherein a plurality of said clamp elements are substantially parallel to each other and joined together at said first end to form a clamp element array, and said linear displacement mechanism includes an externally threaded section of said clamping array engaged with said threaded portion.

38) The implant of claim 36 wherein a second end of said clamping element is attached to said elongated stem portion thereby substantially fixing an axial position of said end of said clamping element.

39) The implant of claim 36 wherein said linear displacement mechanism includes a lock for substantially fixing an axial position of said first end of said clamping element.

40) The implant of claim 36 wherein said linear displacement mechanism includes a linear movable element connected to said first end of a said clamp element via a compressible element and a relationship between a linear displacement of said linear movable element and a linear displacement of said first end of said clamp is determined at least in part by compressive properties of said compressive element.

41) The implant of claim 39 wherein said compressive element includes a spring.
